(19) United States
(12) Patent Application Publication
(10) Pub. No.: US 2011/0045607 A1
(43) Pub. Date: Feb. 24, 2011

(54) PROBE COMPOSITE, METHOD FOR MANUFACTURING THE SAME, METHOD FOR USING THE SAME, AND CONTRAST AGENT INCLUDING THE SAME

(75) Inventors: Pai-Chi Li, Taipei (TW); Churng-Ren Wang, Taipei (TW); Dar-Bin Shieh, Taipei (TW); Chen-Wei Wei, Taipei (TW)

Correspondence Address: NORTH AMERICA INTELLECTUAL PROPERTY CORPORATION, P.O. BOX 506, MERRIFIELD, VA, 22116, US

(73) Assignee: NATIONAL TAIWAN UNIVERSITY, Taipei (TW)

(21) Appl. No.: 12/545,093

(22) Filed: Aug. 21, 2009

(51) Int. Cl. G01N 33/553

(52) U.S. Cl. 436/525

(57) ABSTRACT

A probe composite for photoacoustic imaging includes a first probe and the second probe mixed with each other. The first probe includes a first ligand and a first nanorod conjugated to the first ligand. The first ligand specifically interacts with a first target. The second probe includes a second ligand and a second nanorod conjugated to the second ligand. The second ligand specifically interacts with a second target.

### BACKGROUND

1. Technical Field

The present disclosure relates to probe composites, contrast agents including the same and methods for manufacturing and for using the same, and particularly, to a probe composite, a contrast agent including the same, and a method for manufacturing or using the same for photoacoustic imaging.

2. Description of Related Art

Photoacoustic imaging is an imaging modality developed based on the photoacoustic effect. Currently, photoacoustic imaging is employed in several biomedical applications for obtaining anatomic and bio-function information.

Photoacoustic imaging is accomplished by transforming optical energy into acoustic energy and the subsequent outward propagation of an acoustic wave. In practice, optical energy, generally from a laser pulse, is delivered into biological tissue. Some of delivered energy will be absorbed by the tissue and converted to heat, leading to transient thermo-elastic expansion and thus ultrasonic emission. The generated ultrasonic waves are then detected by ultrasonic transducers to form images. The magnitude of ultrasonic emission can be interpreted to reveal physiological information of the tissue because the varying amounts of optical absorption can be correlated to physiological properties, such as glucose concentration of the tissue. Thus, bio-functional information of tissue can be acquired.

Physiologically, aberrations at the cellular and molecular levels occur much earlier than more apparent gross anatomical changes in tissue. For achieving early detection and more effective treatment of diseases, molecular imaging methods have been developed for various imaging modalities. For example, a specific probe having a high affinity to a specific biomarker may be used to sense the characteristics of a particular biological process at the molecular level to detect presence and/or progress of a specific disease, because the expression of the specific biomarker is highly associated with the specific disease. Thus, molecular imaging also can be used to aid in predicting clinical outcome and treatment responses. However, the conventional molecular imaging methods employed are only capable of targeting a single biomarker or disease. For obtaining more reliable and accurate diagnostic results, there is a demand for developing photoacoustic imaging capable of targeting multiple biomarkers to acquire more detailed and diverse bio-function information of tissue at the same time.

### BRIEF DESCRIPTION OF THE DRAWINGS

Many aspects of the embodiments can be better understood with references to the following drawings. The components in the drawings are not necessarily drawn to scale, the emphasis instead being placed upon clearly illustrating the principles of the present probe composite, method for manufacturing the same, method for using the same and contrast agent including the same.

FIG. 1 is a schematic view of one embodiment of a probe composite.

FIG. 2 is a schematic view showing absorption spectra of gold nanorods with different aspect ratios.

FIG. 3 is a flow chart of one embodiment of a method for manufacturing the probe composite of FIG. 1

FIG. 4 is a flow chart of one embodiment of a method for using the probe composite of FIG. 1.

FIG. 5 is a schematic view illustrating the use of the probe composite of FIG. 1 to detect at least two targets in a biological sample.

Corresponding reference characters indicate corresponding parts throughout the several views. The exemplifications set out herein illustrate at least one embodiment of the present heat probe composite, method for manufacturing the same, method for using the same and contrast agent including the same, in one form, and such exemplifications are not to be construed as limiting the scope of the disclosure in any manner.

### DETAILED DESCRIPTION

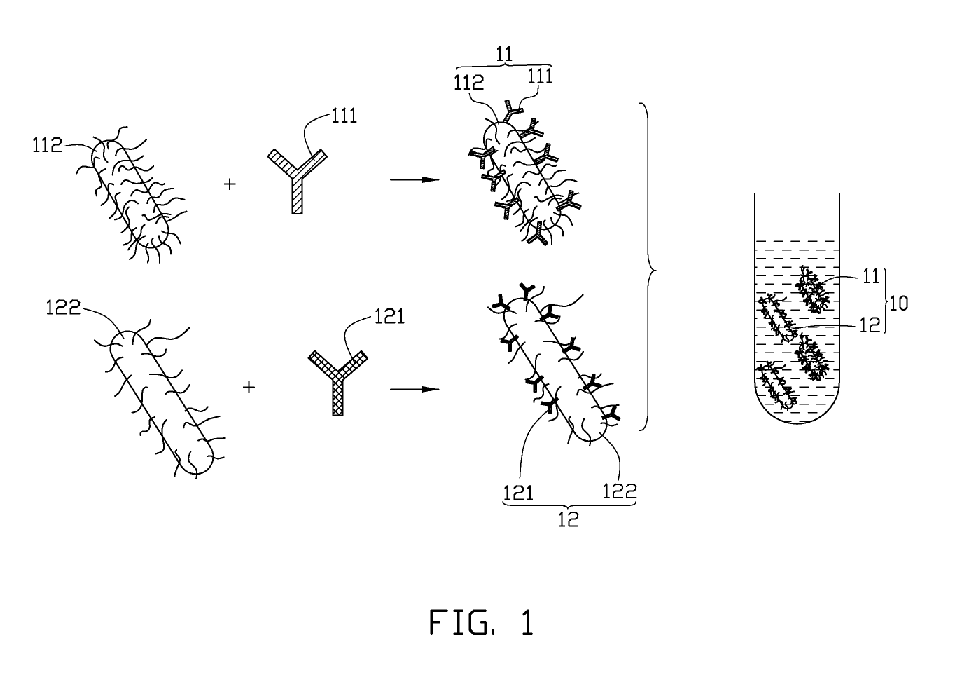

Referring to FIG. 1, one embodiment of a probe composite 10 includes a combination of a first probe 11 and a second probe 12. The first probe 11 includes a first ligand 111 and a first nanorod 11 2 conjugated to the first ligand 111. The second probe 12 includes a second ligand 121 and a second nanorod 122 conjugated to the second ligand 121. In the present embodiment, the probe composite 10 is employed in photoacoustic imaging for detecting at least two targets in a biological tissue simultaneously.

Targets refer to biological molecules, particles or substances, such as antigens, receptors or integrins, serving as indicators for diagnosis or detection of a biological ailment. It is understood that expression of a specific biological substance can indicate characteristics of a specific type of cell or clinical and pathologic feature/response. In the present embodiment, the biological tissue including different oral cancer cells, such as oral squamous cell carcinoma (OECM1) or squamous cell carcinoma (Cal 27), are taken as an example. In addition, epidermal growth factor receptor (EGFR) is taken as a first target of the present embodiment, because its expression is strongly correlated with tumor metastasis. Human epidermal growth factor receptor 2 (HER2) is taken as a second target of the present embodiment, because its expression is associated with growth characteristics and sensitivity to Herceptin chemotherapy. The first probe 11 is configured to specifically tag the first target, i.e. EGFR, while the second probe 12 is configured to specifically tag the second target, i.e. HER2.

The first ligand 111 of the first probe 11 and the second ligand 121 of the second probe 12 are molecules serving to interact with the different targets. That is, the cancer cell is targeted by virtue of specific interaction between, for example, ligands and biologic molecules. The first ligand 111 and the second ligand 121 can be any various ligands according to practical needs, such as antibodies or peptides. In the present embodiment, the first ligand 111 is an EGFR antibody while the second ligand 121 is an HER2 antibody.

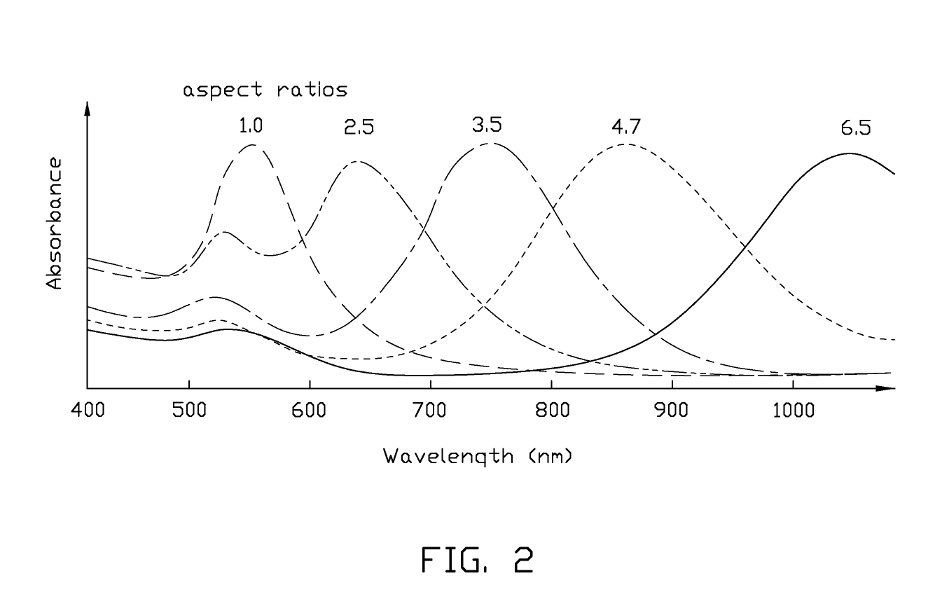

The first nanorod 112 of the first probe 11 and the second nanorod 122 of the second probe 12 are nanostructured material, which are synthesized by an electrochemical method. The first nanorod 112 and the second nanorod 122 are configured to respectively label the first ligand 111 and the second ligand 121. In the present embodiment, the nanorods 112, 122 are gold nanorods (AuNRs). Particularly, the AuNRs can be served as labeling markers for probes because they exhibit different surface plasmon resonances according to their aspect ratios (i.e. a ratio of its longer dimension to its shorter dimension). That is, optical absorption spectrum of AuNR is aspect ratio dependent. Particularly, the wavelength at which the optical absorption of AuNR is increased, is closely related to their aspect ratio, as shown in FIG. 2. By way of choosing suitable aspect ratios of AuNRs to tag ligands, the ligands can be distinguished by detecting different optical absorption spectra of AuNRs. In the present embodiment, the AuNR is chosen to have a peak absorption ranging from about 500 nanometers (nm) to about 1100 nm. For example, the first nanorod 112 is chosen to have an aspect ratio of about 5.9, with a corresponding peak absorption of about 1000 nm. The second nanorod 122 is chosen to have an aspect ratio of about 3.7, with a corresponding peak absorption of about 800 nm.

In the present embodiment, the first ligand 111 and the second ligand 121 are respectively and stably conjugated to the first nanorod 112 and the second nanorod 122 through chemical bonding. Furthermore, in order to avoid nonspecific binding to the desired targets, such as electrostatic binding or endocytosis, at least one blocker (PEG) is conjugated to AuNRs with different aspect ratios.

Additionally, one embodiment of a contrast agent includes a probe composite. In the present embodiment, the compositions, features and functions of the probe composite are the same as the probe composition mentioned above. Thus, the detailed description of the probe composition in the present embodiment is omitted for conciseness. The contrast agent of the present embodiment is used in the photoacoustic imaging to enhance the contrast of targets within the biological tissue.

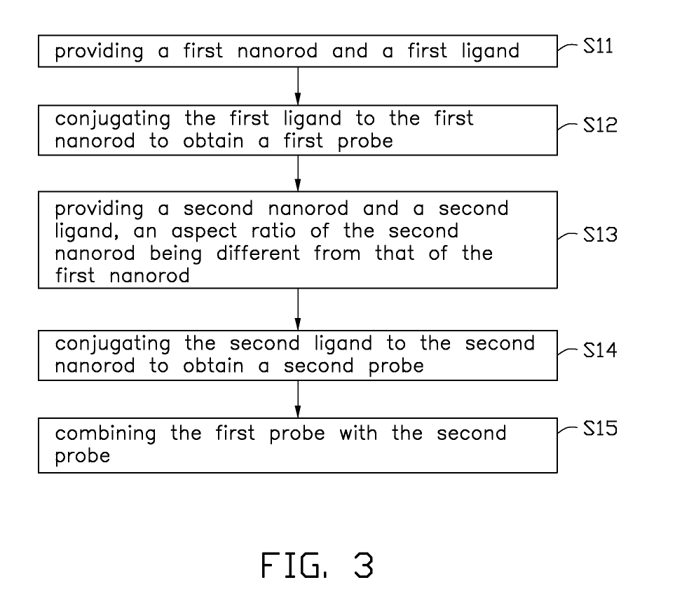

Referring to FIG. 1 and FIG. 3, one embodiment of a method for manufacturing a probe composite 10 is shown. Step S11 includes providing a first nanorod 112 and a first ligand 111. In step S12, the first ligand 111 is conjugated to the first nanorod 112 to obtain a first probe 11. In step S13, a second nanorod 122 and a second ligand 121 are provided. In step S14, the second ligand 121 is conjugated to the second nanorod 122 to obtain a second probe 12. In step S15, the first probe 11 and the second probe 12 are combined.

The method is described in more detail as follows.

In step S11, the first nanorod 112 can be a gold nanorod (AuNR) having a surface plasmon resonance of about 1000 nm while the first ligand 111 can be an EGFR antibody. The AuNR is synthesized by the electrochemical conversion of an anodic gold material into particles within an electrolytic cosurfactant system. In the electrolytic cosurfactant system, the cationic surfactants can be hexadecyltrimethylammonium bromide (CTABr) and tetradodecylammonium bromide (TDABr). The particle shape is successfully controlled using cationic cosurfactant micelles that include several other ingredients, such as cyclohexane and a trace amount of silver ions. The synthesized AuNRs are then well dispersed in aqueous solutions.

In step S12, the first ligand 121 and the first nanorod 122 are conjugated to each other by a conjugation protocol involving a 1-ethyl-3-(3-dimethylaminopropyl)carbodiimide hydrochloride (EDC-mediated coupling reaction). The first probe 11, i.e. AuNR-EGFR, is obtained accordingly.

In step S13, the second nanorod 122 can be an AuNR having a surface plasmon resonance at about 800 nm while the second ligand 121 can be a HER2 antibody. It is noted that an aspect ratio of the second nanorod 122 is different from that of the first nanorod 112, thus allowing a peak absorption of the second nanorod 122 to be different from that of the first nanorod 112.

In Step S14, the second ligand 121 and the second nanorod 122 can be conjugated to each other by a conjugation method employed as in step S12. Thus, the second probe 12, i.e. AuNR-HER2, is formed by conjugating the second ligand 121 and second nanorod 122.

In step S15, the first probe 11 and the second probe 12 are combined in a solution to finally obtain the probe composite 10 for photoacoustic imaging.

In the present embodiment, the method may further include a step of attaching at least one blocker to the first nanorod 112 or the second nanorod 122. A blocking agent, which can be thiol-terminated methoxypoly (ethyleneglycol), mPEG-SH, is used to conjugate the blocker at a nonspecific adsorption site on the AuNRs. As a result, specificity between the probes 11, 12 and the targets is improved.

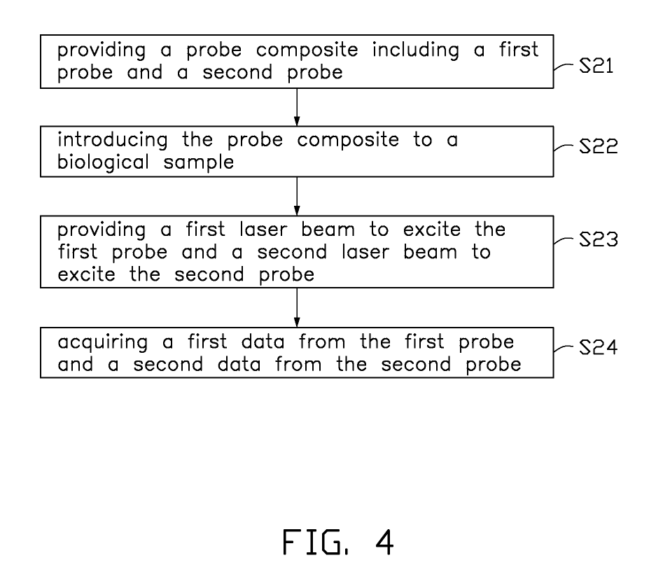

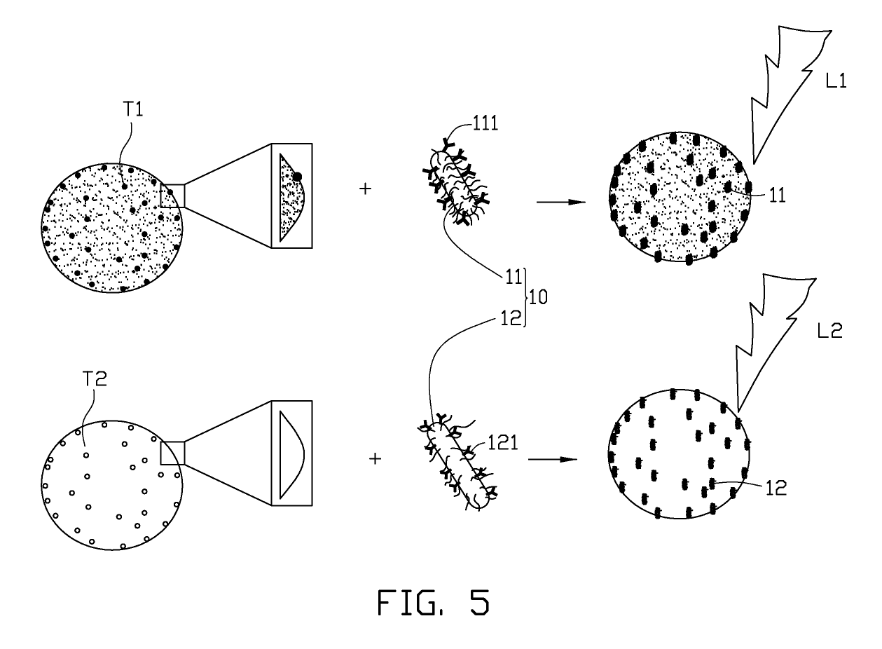

Referring to FIG. 4 and FIG. 5, one embodiment of a method for using a probe composite 10 to detect at least two targets T1, T2 in a biological sample is shown. Step S21 includes providing a probe composite 10 including a first probe 11 and a second probe 12 combined therein. In step S22, the probe composite 10 is introduced to the biological sample. In step S23, a first laser beam L1 is provided to excite the first probe 11 and a second laser beam L2 is provided to excite the second probe 12, wherein a wavelength of the first laser beam L1 is different from that of the second laser beam L2. In step S24, first data from the first probe 11 and second data from the second probe 12 are acquired.

The method is described in more detail as follows.

In the present embodiment, the probe composite 10 is used to detect the cancer associated markers in the biological sample, such as animal or human, based on photoacoustic effect. In step S21, the probe composite 10 of the present embodiment is the same as the probe composite described above. The first probe 11 is designed to tag a first target T1, for example, EGFR expression and the second probe 12 is designed to tag a second target T2, for example, HER2 expression in the biological sample.

In step S22, the probe composite 10, having a combination of the first probe 11 and the second probe 12, is introduced, for example, via injection, in the biological sample. The introduced first probe 11 targets the EGFR specific to the first ligand 111 of the first probe 11. The introduced second probe 12 targets the HER2 specific to the second ligand 121 of the second probe 12.

In step S23, a tunable pulsed Ti:sapphire laser is used to emit the first laser beam L1 and the second laser beam L2 in a wide range of wavelengths. It is noted that the laser beams L1, L2 of the present embodiment are chosen according to the peak absorption wavelengths of the AuNR. In the present embodiment, the first laser beam L1 with laser pulses at about 1000 nm and the second beam L2 with laser pulses at about 800 nm are provided to irradiate the biological sample. Particularly, the AuNR-EGFR will be excited by the laser beam L1 at about 1000 nm and the AuNR-HER2 will be excited by the laser beam L2 at about 800 nm. Accordingly, the two probes 11, 12, i.e. AuNR-EGFR and AuNR-HER2, will generate photoacoustic signals (PA signals).

In step S24, the first data from the first probe 11, i.e. the PA signal from AuNR-EGFR, and the second data from the second probe 12, i.e. the PA signal from AuNR-HER2, are detected by a PA transducer to form different images. Accordingly, the tumor regions in the biological sample can be defined. Furthermore, different bio-functional information can be obtained by way of utilizing wavelength selectivity of PA detection. In the present embodiment, the PA transducer is made of lithium niobate (LiNbO₃) material with a center frequency of about 20 MHz and a focal depth of about 9.5 mm.

In conclusion, the probe composite of the present embodiment is employed in photoacoustic imaging and allows such imaging modality to have an ability to detect multiple selective targets simultaneously by way of utilizing the nanorods with different aspect ratios to serve as labeling markers. Consequently, different types of biological tissues, for example, cancer cells, can be recognized and multiple characteristics can be obtained easily. Moreover, employment of probe composites makes the detection in the photoacoustic imaging at molecular level possible. Thus, besides anatomic information, molecular bio-information of the detected biological sample can also be obtained by photoacoustic imaging employing the probe composite of the present embodiment.

Finally, it is to be understood that the above-described embodiments are intended to illustrate rather than limit the disclosure. Variations may be made to the embodiments without departing from the spirit of the disclosure as claimed. The above-described embodiments illustrate the scope of the disclosure but do not restrict the scope of the disclosure.

It is also to be understood that above description and the claims drawn to a method may include some indication in reference to certain steps. However, the indication used is only to be viewed for identification purposes and not as a suggestion as to an order for the steps.

What is claimed is:

1. A probe composite for photoacoustic imaging, comprising:
a first probe comprising a first ligand and a first nanorod conjugated to the first ligand, the first ligand specifically interacting with a first target; and
a second probe combined with the first probe, the second probe comprising a second ligand and a second nanorod conjugated to the second ligand, the second ligand specifically interacting with a second target.

2. The probe composite of claim 1, wherein an aspect ratio of the first nanorod is different from that of the second nanorod.

3. The probe composite of claim 2, wherein the first nanorod or the second nanorod has a peak absorption in a range from about 500 nm to about 1100 nm.

4. The probe composite of claim 1, wherein the first nanorod or the second nanorod is a gold nanorod.

5. The probe composite of claim 1, wherein the first ligand or the second ligand is a molecule selected from the group consisting of antibody, peptide, and combination thereof.

6. The probe composite of claim 1, wherein the first target or the second target is a molecule selected from the group consisting of antigen, receptor, and integrin.

7. The probe composite of claim 1, further comprising at least one blocker conjugated to one of the first nanorod or the second nanorod.

8. A contrast agent for photoacoustic imaging, comprising:
a probe composite comprising a first probe and a second probe mixed with each other, the first probe comprising a first ligand and a first nanorod conjugated to the first ligand, and the second probe comprising a second ligand and a second nanorod conjugated to the second ligand.

9. The contrast agent of claim 8, wherein the first ligand or the second ligand is an antibody specifically interacting with a receptor.

10. The contrast agent of claim 8, wherein the first nanorod or the second nanorod is a gold nanorod.

11. A method for manufacturing a probe composite for photoacoustic imaging, comprising:
providing a first nanorod and a first ligand;
conjugating the first ligand to the first nanorod to obtain a first probe;
providing a second nanorod and a second ligand, an aspect ratio of the second nanorod being different from that of the first nanorod;
conjugating the second ligand to the second nanorod to obtain a second probe; and
combining the first probe with the second probe.

12. The method of claim 11, wherein the first nanorod or the second nanorod is a gold nanorod.

13. The method of claim 11, wherein a peak absorption of the first nanorod is different from that of the second nanorod.

14. The method of claim 13, wherein the first nanorod or the second nanorod has a peak absorption in a range from about 500 nm to about 1100 nm.

15. The method of claim 11, wherein the first ligand or the second ligand is a molecule selected from the group consisting of antibody, peptide, and combination thereof.

16. The method of claim 11, further comprising attaching at least one blocker to the first nanorod or the second nanorod.

17. A method for using a probe composite to detect at least two molecular targets in a biological sample, the method comprising:
providing the probe composite, the probe composite comprising a first probe and a second probe combined with the first probe, the first probe comprising a first ligand and a first nanorod conjugated to the first ligand, the second probe comprising a second ligand and a second nanorod conjugated to the second ligand;
introducing the probe composite to the biological sample;
providing a first laser beam to excite the first probe and a second laser beam to excite the second probe, a wavelength of the first laser beam being different from that of the second laser beam; and
acquiring first data from the first probe and second data from the second probe under a photoacoustic effect.

18. The method of claim 17, wherein the first nanorod or the second nanorod is a gold nanorod; and a peak absorption of the first nanorod is different from that of the second nanorod.

19. The method of claim 17, wherein wavelengths of the first laser beam and the second laser beam are in a range from about 500 nm to about 1100 nm.

20. The method of claim 17, wherein the first ligand or the second ligand is an antibody specifically interacted with one of the at least two molecular targets.
